(19) United States
(12) Patent Application Publication
(10) Pub. No.: US 2022/0380304 A1
(43) Pub. Date: Dec. 1, 2022

(54) NOVEL METHOD FOR PRODUCING PERAMIVIR TRIHYDRATE, AND WATER-BASED DRYING THEREOF

(71) Applicant: CKD BIO CORPORATION, Seoul (KR)

(72) Inventors: Hee Un KIM, Chungcheongnam-do (KR); In Kyu KIM, Seoul (KR); Myeong Seok LEE, Chungcheongnam-do (KR); Heung Mo KANG, Chungcheongnam-do (KR); Sae Jin KIM, Gyeonggi-do (KR); In Suk CHOI, Seoul (KR)

(21) Appl. No.: 17/622,322

(22) PCT Filed: Jul. 2, 2020
(86) PCT No.: PCT/KR2020/008624

§ 371 Date: Dec. 23, 2021

(30) Foreign Application Priority Data
Jul. 3, 2019 (KR) 10-2019-0080188

(51) Int. Cl. C07C 277/08

(52) U.S. Cl. CPC C07C 277/08

(57) ABSTRACT

The present invention relates to a method for producing peramivir trihydrate, which is an inhibitor of neuraminidase infection, as an anti-influenza agent. According to the production method of the present invention, peramivir trihydrate can be produced with high yield and stability through a process suitable for producing excellent pharmaceuticals and quality control standards (GMP) without using highly-toxic methanol and activated carbon.

### TECHNICAL FIELD

This application claims priority to and the benefit of Korean Patent Application No. 10-2019-0080188 filed in the Korean Intellectual Property Office on 3 Jul. 2019, the disclosure of which is incorporated herein by reference.

The present invention relates to a method for producing peramivir trihydrate, which is a neuraminidase infection inhibitor as an anti-influenza agent.

### BACKGROUND ART

Influenza viruses cause acute respiratory illnesses as infectious viral diseases and are viruses causing diseases commonly known as the flu. Influenza viruses were first isolated from swine by Shope in 1931 and isolated from humans and known by Smith, Andrewes and Laidlaw in 1933. Currently, influenza viruses are controlled as viruses that cause various acute respiratory diseases in humans worldwide through several times of mutations to result in increases in prevalence and mortality. Recently, the emergence of new viruses through various mutations of influenza viruses is predicted to greatly increase prevalence and mortality, and thus influenza surveillance systems are operated around the world due to the recognition of the importance and severity of influenza viruses.

Influenza virus is an RNA virus belonging to the family Orthomyxoviridae and has a polymorphic spherical shape, with a diameter of about 80-120 nm, composed of eight segments. Influenza virus has hemagglutinin (HA) protein and neuraminidase (NA) protein, which are two glycoproteins, and matrix protein (M2) in the envelope and another matrix protein (M1) in the inner interface of the envelope. Influenza viruses are classified into types A, B, and C according to the antigen type. Influenza A viruses are determined as subtypes thereof on the basis of the surface antigens HA and NA. HA allows viruses to be attach to somatic cells and has H1 to H18 subtypes. NA plays a key role in the release of viruses from infected cells and the propagation thereof to new respiratory cells, and has N1 to N11 subtypes. In contrast, influenza B viruses are smaller in antigenic change than influenza A viruses and have an immunologically stable form. In addition, as described above, humans are the only one host for influenza B viruses, which are divided into Victoria and Yamagata lineages. Last, influenza C causes asymptomatic infections in most cases and is known to be not associated with influenza epidemic.

Anti-influenza infection inhibitors, Oseltamivir (Tamiflu™), Zanamivir (Relenza™), and Peramivir (Peramiflu™) were developed as treatments for preventing such influenza viruses and inhibiting the proliferation of the viruses, and these all have antiviral effects against influenza A and B viruses. However, the M2 inhibitors Amantadine and Rimantadine are effective for only influenza A viruses and are not recommended at present due to the resistance thereof. Oseltamivir, which is an inhibitor of influenza A and B virus infections, shows an effect against influenza viruses by blocking an enzyme for virus reproduction, and should be taken within 48 hours of onset of symptoms for maximum effects thereof. However, oseltamivir must be taken for 5 days even after symptom alleviation, and in recent years, vomiting, nausea, and serious side effects due to neuropsychiatric abnormalities occurred after a dose of oseltamivir, resulting in social problems. Zanamivir, which is another inhibitor for influenza A and B virus infections, is a viral treatment that is not for oral use but is sprayed into the mouth and inhaled. Zanamivir is also known as a treatment showing effects against all of various mutant influenza viruses by inhibiting the proliferative enzyme neuraminidase on the viral envelope to prevent the viral proliferation into other cells. However, zanamivir also has serious side effects, such as diarrhea, nausea, vomiting, headache, dizziness, and nasal bleeding, and the administration thereof is possible for only those over 7 years of age. As for their global marketability, the market of zanamivir is being eroded, and the market of oseltamivir is predicted to be also highly likely to be eroded in that oseltamivir has serious side effects, such as serious neuropsychiatric abnormalities, is difficult to prescribe for patients who have difficulty in oral administration, and should be orally taken about 10 times over 5 days. On the contrary, peramivir is a type of neuraminidase inhibitor that has been developed most recently and is known as a treatment for influenza A and B virus infections. Peramivir has a mechanism of action that inhibits the actions of a wide range of influenza virus strains and can be prescribed with a single intravenous injection. Peramivir is evaluated as a very effective treatment due to its intravenous prescription compared with oseltamivir through oral administration and zanamivir through inhale administration, and can be prescribed for patients who have difficulty in oral administration, patients with metabolic diseases, and patients with chronic respiratory diseases. In addition, the intravenous prescription of peramivir shows faster fever removal and relatively fewer side effects, such as vomiting and nausea, compared with oral description. Peramivir as an influenza infection inhibitor has been showing continuous growth through improvement in awareness after FDA approval in 2014, and the enlargement of the demand class of peramivir is gradually increasing due to the expansion of indications in Korea in September, 2018.

The product name of peramivir is Peramiflu™, which is completely prepared as an injection in the form of a peramivir hydrate as shown in Chemical Formula 1. More specifically, Peramiflu™ has a form of peramivir trihydrate as shown in Chemical Formula 2.

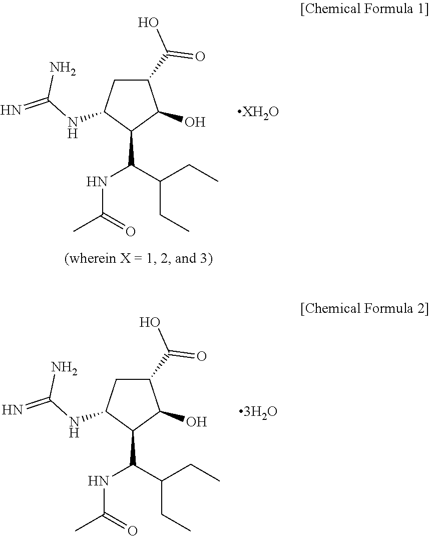 [SMILES: CCC(CC)C(NC(C)=O)[C@@H]1[C@H](O)[C@@H](C(=O)O)C[C@H]1NC(=N)N ; CCC(CC)C(NC(C)=O)[C@@H]1[C@H](O)[C@@H](C(=O)O)C[C@H]1NC(=N)N]

Patents regarding methods for producing peramivir trihydrate are disclosed in KR1020017016653/US2000016013/WO200100571 by the original developer and KR1020107005427/CN2008001459/WO2009021404 and KR1020127025551/CN2008001459/WO2009021404 filed in China.

The method for producing peramivir trihydrate disclosed in KR1020017016653/US2000016013/WO200100571 is shown in Production Process Diagram 1 below, and the methods for producing a trihydrate disclosed in KR1020107005427/CN2008001459/WO2009021404 and KR1020127025551/CN2008001459/WO2009021404 are shown in more detail in Production Process Diagram 2 below.

As for the patent in KR1020017016653/US2000016013/WO200100571 by the original developer, as shown in production process diagram 1, peramivir trihydrate is produced using activated carbon and methanol with intrinsic toxicity, which belongs to Class 2 solvents in pharmaceutical products among solvents of which the residual amounts should be regulated, wherein after the preparation of a peramivir trihydrate, natural drying is employed in drying to the trihydrate, and thus the peramivir trihydrate is produced by a drying process, which is not suitable for the application to industrial production, the manufacture of excellent pharmaceutical products, and good manufacturing practice (GMP).

The present inventors, as a result of conducting vacuum drying in a state without a water system in a drying machine, instead of natural drying, identified a moisture value close to an anhydride but not a trihydrate, and therefore, the production methods of existing patents are supposed to select natural drying. However, peramivir trihydrate is a pharmaceutical product that is completely prepared as an injection, and natural drying requires different drying conditions according to the internal humidity, so that it is difficult to maintain homogeneous moisture values. Therefore, natural drying is not suitable for industrial mass production and GMP standards.

Like in the patents in KR1020107005427/CN2008001459/WO2009021404 and KR1020127025551/CN2008001459/WO2009021404 shown in Production Process Diagram 2, a trihydrate is prepared using a methanol solvent, and peramivir trihydrate is produced through a process that is not easy for GMP regulations and industrial application. In both of Production Process Diagrams 1 and 2, a trihydrate is produced in the presence of activated carbon. The industrial yields thereof from peramivir to peramivir trihydrate are 73.1% and 90%, respectively, and the purities of peramivir trihydrate are 99.91% and 99.86%, respectively, and the products through these patents have moisture values in the range of 14%.

Therefore, the present inventors conducted intensive research efforts to develop a production method suitable for the manufacture of excellent pharmaceutical products and good manufacturing practice (GMP) even without the use of toxic solvents, such as activated carbon and methanol, in the production of peramivir trihydrate.

### SUMMARY

### Technical Problem

The present invention has been made to provide a trihydrate production technique for avoiding the use of methanol with intrinsic toxicity belonging to Class 2 solvents in pharmaceutical products, of which the residual amounts should be regulated, and for excluding the use of activated carbon in the production of peramivir trihydrate completely prepared as an injection.

An aspect of the present invention is to provide a trihydrate of chemical formula 2, which has low toxicity and is produced with an improved synthesis yield compared with the conventional art, by using an alcoholic solvent, such as 1-propanol, 2-propanol, 1-pentanol, 2-butanol, 3-methyl-1-butanol, 2-methyl-1-propanol, belonging to Class 3 solvents in pharmaceutical products, in the production of peramivir trihydrate.

Another aspect of the present invention is to provide a method for producing peramivir trihydrate of chemical formula 2, which is prepared by vacuum drying of raw materials and a water system through a drying process which is easy for the industrial production and suitable for the manufacture of excellent pharmaceutical products and the good manufacturing practice (GMP) standards.

A final aspect of the present invention is to provide peramivir trihydrate having a high yield and a low level of a residual solvent compared with the conventional art (methanol) according to the above-described production and water system drying process.

### Solution to Problem

A process for producing peramivir trihydrate according to the present invention is shown in Reaction Scheme 1 below.

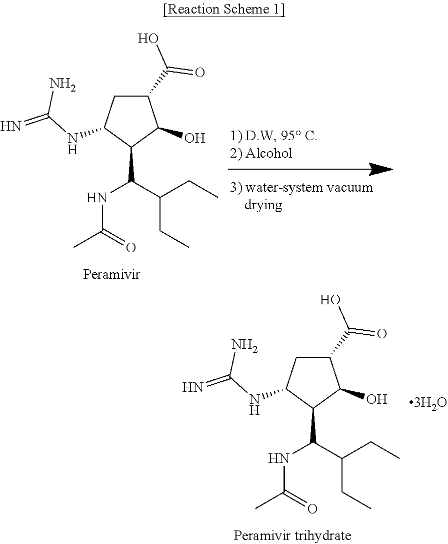 [SMILES: CCC(CC)C(NC(C)=O)[C@@H]1[C@H](O)[C@@H](C(=O)O)C[C@H]1NC(=N)N ; CCC(CC)C(NC(C)=O)[C@@H]1[C@H](O)[C@@H](C(=O)O)C[C@H]1NC(=N)N]

In the process for producing peramivir trihydrate according to the present invention, a low toxic alcoholic solvent belonging to Class 3 solvents in pharmaceutical products is used instead of a methanol solvent with intrinsic toxicity belonging to Class 2 solvents in pharmaceutical products to be regulated in the manufacture of pharmaceutical products.

The solvents belonging to Class 2 to be regulated in the manufacturing of pharmaceutical products are solvents of which the residual amounts should be regulated, and mean solvents showing no genotoxicity but exhibiting carcinogenicity in animal tests, solvents showing irreversible toxicity other than carcinogenicity, such as neurotoxicity and teratogenicity, and other solvents suspected of serious but reversible toxicity.

The solvents belonging to Class 3 to be regulated in the manufacturing of pharmaceutic products are low toxic solvents, and mean solvents that are considered to be low toxic to humans and solvents of which the exposure limit concentration need not be established for health reasons.

According to an aspect of the present invention, there is provided a method for producing (1S,2S,3R,4R)-3-[(S)-1-acetylamido-2-ethylbutyl]-4-guanidino-2-hydroxycyclopentane-1-carboxylic acid trihydrate (peramivir trihydrate) represented by chemical formula 2:

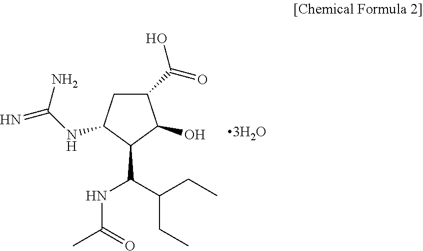 [SMILES: CCC(CC)C(NC(C)=O)[C@@H]1[C@H](O)[C@@H](C(=O)O)C[C@H]1NC(=N)N]

the method comprising: dissolving (1S,2S,3R,4R)-3-[(S)-1-acetylamido-2-ethylbutyl]-4-guanidino-2-hydroxycyclopentane-1-carboxylic acid (peramivir) in water to prepare an aqueous solution; and

adding an alcoholic solvent selected from 1-propanol, 2-propanol, 1-pentanol, 2-butanol, 3-methyl-1-butanol, 2-methyl-1-propanol, or a mixture thereof.

In an embodiment of the present invention, the peramivir and water are added such that the weight of water to the weight of peramivir is 1:1 to 1:30, specifically 1:1 to 1:20, 1:1 to 1:15, 1:1 to 1:10, 1:1 to 1:8, 1:3 to 1:10, 1:5 to 1:10, 1:8 to 1:10, 1:3 to 1:8, or 1:5 to 1:8, and most specifically 1:8. The addition of peramivir and water at a weight ratio of water to peramivir of 1:8 leads to a highest synthesis yield of a final product.

In another embodiment of the present invention, the dissolving is conducted with heating. A mixture of peramivir and water is in a state of a suspension at the initial stage, and thus it is preferable to increase the solubility through heating.

In a specific embodiment of the present invention, the heating may be conducted at a temperature of 50 to 100° C., more specifically 60-100° C., 70-100° C., 80-100° C., 90-100° C., 60-95° C., 70-95° C., 80-95° C., and most specifically 90-95° C. The heating conducted at a temperature of 90 to 95° C. can completely dissolve peramivir in a state of a suspension of peramivir and water and minimize the loss of evaporated purified water. The heating conducted at a temperature of 95° C. or higher results in the evaporation of a portion of added water (purified water), so that as much water (purified water) as the water that is evaporated needs to be supplemented, and therefore, such heating is unsuitable for industrial mass production.

As described above, the production method of the present invention comprises adding an alcoholic solvent selected from 1-propanol, 2-propanol, 1-pentanol, 2-butanol, 3-methyl-1-butanol, 2-methyl-1-propanol, or a mixture thereof.

In the production method of the present invention, the alcoholic solvent in Reaction Scheme 1 corresponds to a low toxic solvent belonging to Class 3 to be regulated in the manufacture of pharmaceutical products.

In a specific embodiment of the present invention, the use of 2-propanol as the alcoholic solvent results in a highest synthesis yield of peramivir trihydrate and thus is preferable.

In another embodiment of the present invention, the alcoholic solvent is added at a volume ratio, relative to the volume of water, of 1:1-10 (alcoholic solvent:water). More specifically, the alcoholic solvent is added at a volume ratio, relative to the volume of water, of 1:2-9, 1:3-8, or 1:4-7, but is not limited thereto. Most specifically, the alcoholic solvent is added at a volume ratio, relative to the volume of water, of 1:5-6. According to an exemplary embodiment of the present invention, the addition of the alcoholic solvent at a volume ratio, relative to the volume of water, of 1:5-6 leads to an improvement in the synthesis yield of peramivir trihydrate.

In an embodiment of the present invention, the alcoholic solvent is added at 75 to 90° C. In an exemplary embodiment of the present invention, the alcoholic solvent was added at 85° C., but is not limited thereto.

In another embodiment of the present invention, the production method of the present invention further comprises cooling the aqueous solution with the alcoholic solvent added thereto, to a temperature of 15 to 30° C. The cooling temperature of 15-30° C. may be more specifically 20-30° C., 25-30° C., 15-25° C., or 20-25° C., and most specifically 25° C. In the cooling, peramivir trihydrate crystals are generated. According to an exemplary embodiment, peramivir trihydrate crystal grains start to generate from about 40° C.

In an embodiment of the present invention, the production method further comprises performing stirring for 6 to 24 hours after the addition of the alcoholic solvent and the cooling. The stirring time may be more specifically 6-24 hours, 8-24 hours, 10-24 hours, 12-24 hours, 6-18 hours, 8-18 hours, 10-18 hours, 6-12 hours, 8-12 hours, or 10-12 hours, and most specifically 12 hours. The stirring is a step for phase transition of the generated peramivir trihydrate crystals to crystal form A. The crystallization process by continuous stirring for the aforementioned periods of time, compared with the previously disclosed production methods of performing stirring-standing-restirring, enables the omission of standing and restirring, thereby further simplifying processes and generating homogeneous final trihydrate crystals, thus significantly improving filterability of the resultant crystallized product.

In the present invention, the production method may further comprise, after the stirring for phase transition to crystal form A, performing stirring with cooling to a temperature of 1-10° C.

In the present invention, the production method further comprises filtering the generated peramivir trihydrate crystals. Through the filtering, a peramivir trihydrate crystal cake is formed.

In an embodiment of the present invention, the production method of the present invention further comprises washing the peramivir trihydrate crystal cake formed by the filtering with an alcoholic aqueous solution.

In a specific embodiment of the present invention, the alcoholic aqueous solution may be an aqueous solution of 1-propanol, an aqueous solution of 2-propanol, an aqueous solution of 1-pentanol, an aqueous solution of 2-butanol, an aqueous solution of 3-methyl-1-butanol, or an aqueous solution of 2-methyl-1-propanol.

In another specific embodiment of the present invention, the concentration of the alcoholic aqueous solution is 1-30%, 1-20%, 1-10%, 5-30%, 5-20%, 5-10%, or 10%.

In still another specific embodiment of the present invention, the volume of the alcoholic aqueous solution used for the washing may be a volume corresponding to 0.1-20 times, 0.1-15 times, 0.1-10 times, 0.1-5 times, 0.1-3 times, 0.1-1 times, or 0.1-0.5 times, and most specifically 0.5 times the initial weight of peramivir. For example, the volume of the alcoholic aqueous solution used for washing may be 15 L when the weight of peramivir is 30 kg.

According to the conventional art, the peramivir trihydrate crystal cake needs to be left for at least 48 hours at the time of natural drying in order to produce peramivir trihydrate with a homogeneous moisture level in the range of 14%. Such a natural drying method is a drying process that is not suitable for industrial mass production of pharmaceutical products and is not suitable for the manufacture of excellent pharmaceutical products and the good manufacturing practice (GMP) standards.

Therefore, according to an embodiment of the present invention, the method for producing peramivir trihydrate of the present invention further comprises subjecting the peramivir trihydrate crystal cake generated by the above-described process to vacuum drying or decompression drying.

In a specific embodiment of the present invention, the drying may be conducted in a temperature condition of 25 to 80° C., more specifically 25-70° C., 25-60° C., 25-55° C., 25-50° C., 25-45° C., 25-40° C., 30-70° C., 30-60° C., 30-55° C., 30-50° C., 30-45° C., 30-40° C., 35-70° C., 35-60° C., 35-55° C., 35-50° C., 35-45° C., or 35-40° C., and most specifically 40° C.

In another specific embodiment of the present invention, humidification drying may be conducted in a humidity condition of 20-80%, more specifically 20-70%, 20-65%, 20-60%, 30-80%, 30-70%, 30-65%, 30-60%, 30-55%, 30-50%, 30-45%, 30-40%, 40-80%, 40-70%, 40-60%, 40-55%, 40-50%, or 40-45%. Preferably, the humidification drying at a humidity of 40-60% is suitable for maintaining a constant moisture value of peramivir trihydrate, and most preferably, 40% is suitable.

In still another specific embodiment of the present invention, when the alcoholic solvent used in the washing process of the present invention is 2-propanol, drying at a temperature of 40° C. is suitable for maintaining a moisture value of peramivir trihydrate at a range of 14% and meeting the residual solvent acceptance limits for 2-propanol (50 mg/day, 5,000 ppm).

As proved in an exemplary embodiment of the present invention, the peramivir trihydrate prepared by vacuum drying of a raw material with a water system of the present invention was shown to have a moisture of 14.6% and a significantly low level of 2-propanol residual, 262 ppm, suitable within the acceptance limits.

The industrial yields in the trihydrate production methods using methanol in the conventional art patents KR 10-2001-7016653, KR 10-2010-7005427, and KR 10-2012-7025551, were reported to have 73.1% and 90%, respectively. However, as proved in an exemplary embodiment of the present invention, the industrial yield in the use of 2-propanol as an alcoholic solvent of the present invention was observed to be about 95%, indicating a significantly improved synthesis yield of peramivir trihydrate.

Furthermore, the method for producing peramivir trihydrate of the present invention can produce peramivir trihydrate even without using activated carbon, unlike Production Process Diagram 1 or 2 using activated carbon.

A method for producing peramivir trihydrate according to a specific embodiment of the present invention is shown in Production Process Diagram 3.

As set forth above, the production method according to the present invention is suitable and easy for the industrial production of pharmaceutical products and enables production suitable for the manufacture of excellent pharmaceutical products and the good manufacturing practice (GMP) standards.

### Advantageous Effects of Invention

The present invention is directed to a method for producing peramivir trihydrate, which is a neuraminidase infection inhibitor, as an anti-influenza agent, and according to the production method of the present invention, peramivir trihydrate can be produced with a high yield and stability by a process suitable for GMP standards, without the use of highly toxic methanol and activated carbon.

### BRIEF DESCRIPTION OF THE DRAWINGS

FIG. 1 is a schematic diagram for a production process of peramivir trihydrate of the present invention.

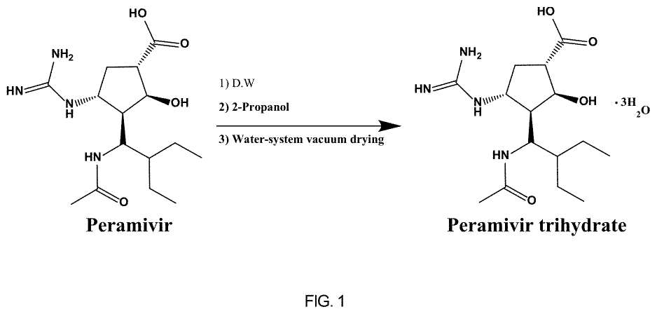

FIG. 2A shows a liquid chromatography (HPLC) spectrum of peramivir trihydrate produced in Example 2 of the present invention.

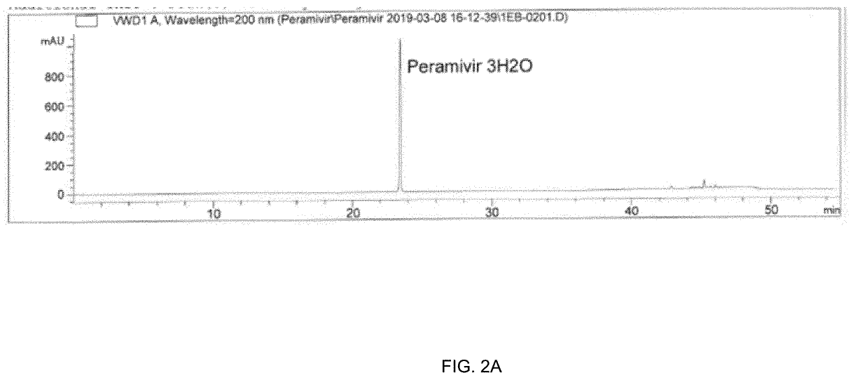

FIG. 2B shows a liquid chromatography (HPLC) spectrum of peramivir trihydrate produced in Example 2 of the present invention.

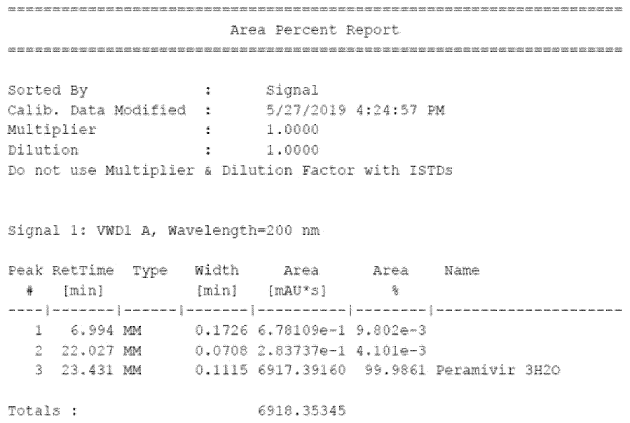

FIG. 3 shows a ¹H-NMR spectrum of peramivir trihydrate, a compound produced in Example 2 of the present invention.

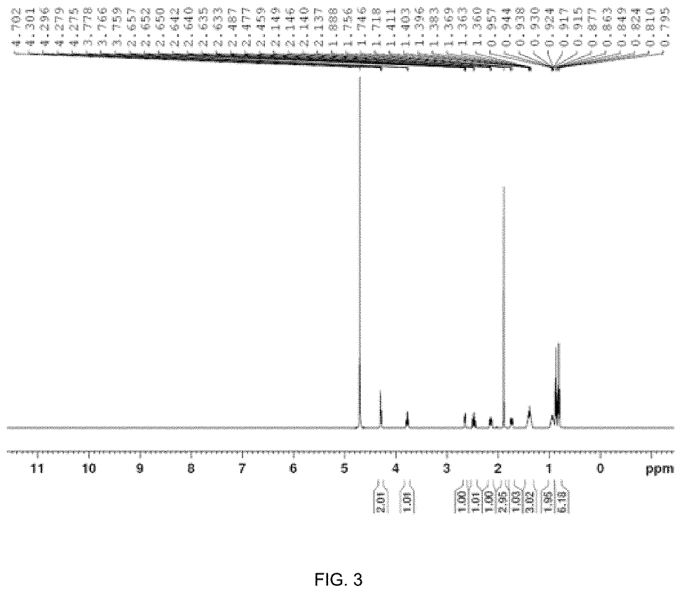

FIG. 4 shows a ¹³C-NMR spectrum of peramivir trihydrate, a compound produced in Example 2 of the present invention.

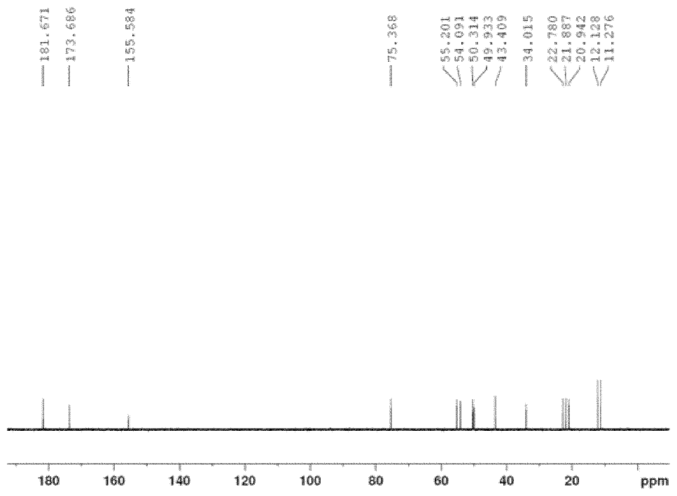

FIG. 5 shows a liquid chromatography-mass spectrometry (LC-MS) of peramivir trihydrate, a compound produced in Example 2 of the present invention.

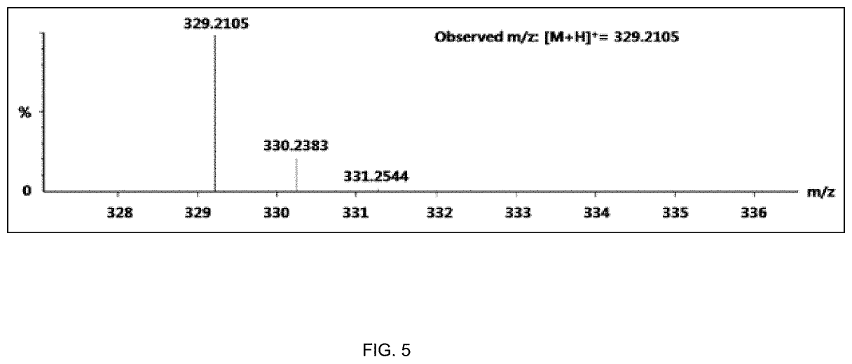

FIG. 6 shows X-ray diffraction (XRD) analysis values of crystal form A of peramivir trihydrate, a compound produced in Example 2 of the present invention.

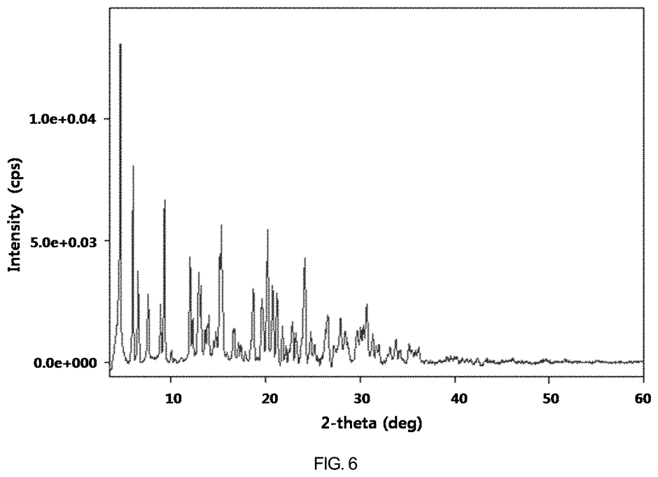

FIG. 7A shows a gas chromatography (GC) spectrum for acceptance limit of each detectable solvents.

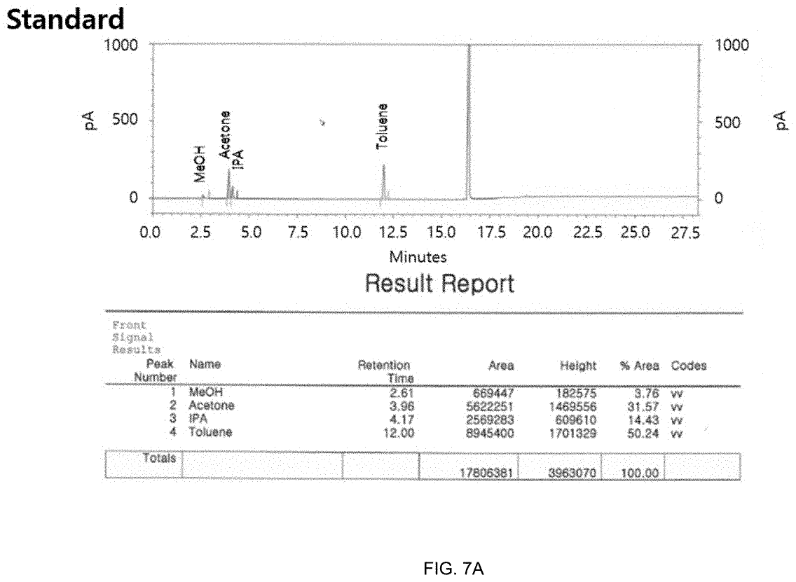

FIG. 7B shows a gas chromatography (GC) spectrum of peramivir trihydrate, a compound produced in Example 2 of the present invention.

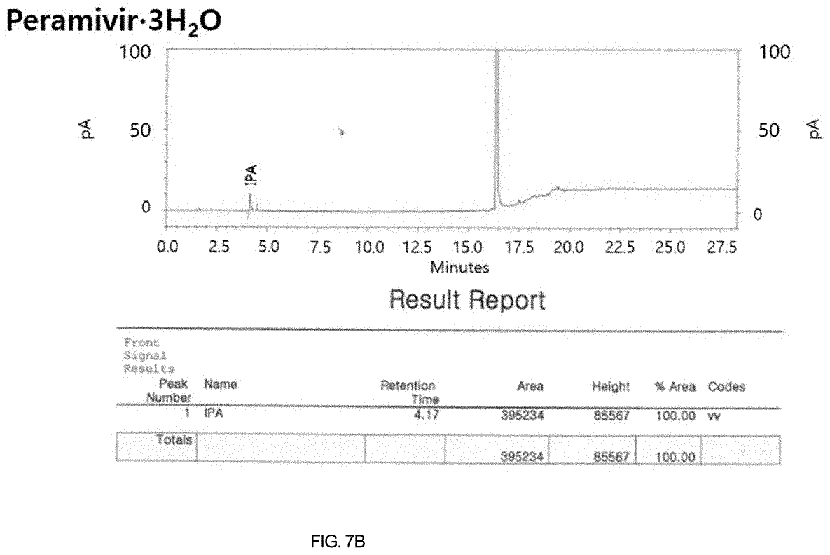

### DETAILED DESCRIPTION

Hereinafter, the present invention will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these examples are not construed to limit the scope of the present disclosure.

### EXAMPLES

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

Reagents and Analysis

Peramivir (1S,2S,3R,4R)-3-[(S)-1-acetylamido-2-ethylbutyl]-4-guanidino-2-hydroxycyclopentane-1-carboxylic acid), purified water, 1-propanol, 2-propanol, 1-pentanol, and the like were used without purification. For the progression of reaction processes and the identification of purity in the present invention, an instrument, such as liquid chromatography (HPLC), was used to carry out analysis and measurement. For the identification of structures and the analysis of crystal forms, moisture, and residual solvent, nuclear magnetic resonance (¹H NMR, ¹³C NMR), liquid chromatography mass spectrometry (HPLC), X-ray diffraction analyzer (XRD), moisture meter (Karl Fischer), and gas chromatography (GC) were used to carry out analysis.

### Example 1: Preparation of Peramivir Trihydrate ((1S,2S,3R,4R)-3-[(S)-1-acetylamido-2-ethyl butyl]-4-guanidino-2-hydroxycyclopentane-1-carboxylic Acid Trihydrate) (1)

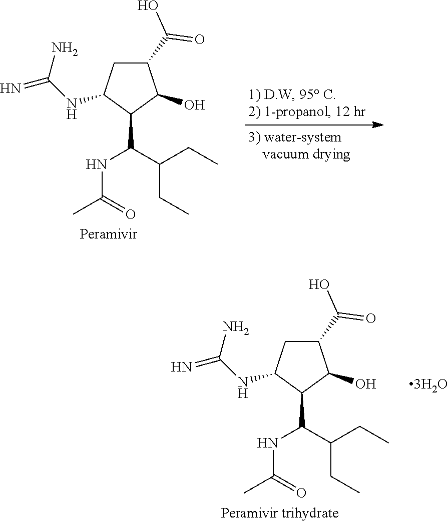 [SMILES: CCC(CC)C(NC(C)=O)[C@@H]1[C@H](O)[C@@H](C(=O)O)C[C@H]1NC(=N)N ; CCC(CC)C(NC(C)=O)[C@@H]1[C@H](O)[C@@H](C(=O)O)C[C@H]1NC(=N)N]

To a 500.0-L reactor, 30.0 kg of (1S,2S,3R,4R)-3-[(S)-1-acetylamido-2-ethylbutyl]-4-guanidino-2-hydroxycyclopentane-1-carboxylic acid (peramivir) was added, and suspended in 240.0 L of purified water. The reaction product was heated at about 95° C. to completely dissolve the suspension, followed by natural cooling with stirring. When the temperature of the reaction product was 85° C., 45.0 L of 1-propanol was slowly added, followed by natural cooling to 25° C. with stirring. Then, peramivir trihydrate crystal grains were generated at about 40° C. When the temperature of the reactor reached 25° C. through natural cooling, stirring was carried out for about 12 hours for phase transition to crystal form A. In addition, the temperature of the reactor was cooled to 5° C., followed by further stirring for about 3 hours, and after the completion of the reaction, filtration was carried out and washing with 15.0 L of 10% 1-propanol solvent was carried out once. The filtered solid cake was placed in a vacuum dryer with an inner humidity of about 40% in which a purified water bowl was placed in the lower portion, and the solid cake was dried at 40° C. for about 12 hours. After the completion of the drying, peramivir trihydrate was obtained (yield: 95.0%, purity: 99.9%, and moisture: 14.5%).

¹H NMR (500 MHz, D₂O, δ): 4.30-4.27 (m, 2H), 3.79-3.74 (m, 1H, —CH), 2.65-2.63 (m, 1H, —CH), 2.48-1.45 (m, 1H, —CH), 2.16-2.12 (m, 1H, —CH), 1.88 (s, 3H, —CH₃), 1.75-1.71 (m, 1H, —CH), 1.41-1.36 (m, 3H), 0.95-0.91 (m, 2H), 0.87-0.84 (t, 3H, —CH₃), 0.82-0.79 (t, 3H, —CH₃); ¹³C NMR (500 MHz, D₂O, δ): 181.671, 173.686, 155.584, 75.368, 55.201, 54.091, 50.314, 49.933, 43.409, 34.015, 22.780, 21.887, 20.942, 12.128, 11.276; LC-MS (m/z, C₁₅H₂₈N₄O₄): calcd for 329.21, found; 329.5.

### Example 2: Preparation of Peramivir Trihydrate ((1 S,2S,3R,4R)-3-[(S)-1-acetylamido-2-ethyl butyl]-4-guanidino-2-hydroxycyclopentane-1-carboxylic Acid Trihydrate) (2)

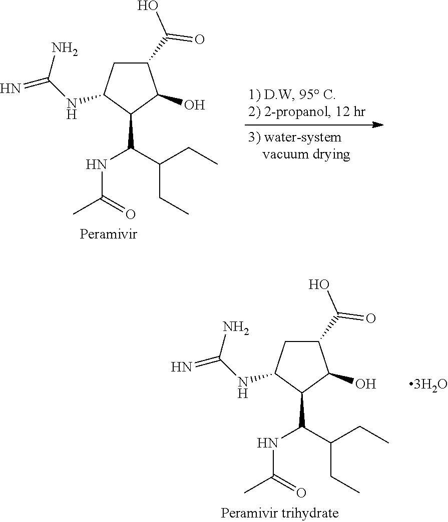 [SMILES: CCC(CC)C(NC(C)=O)[C@@H]1[C@H](O)[C@@H](C(=O)O)C[C@H]1NC(=N)N ; CCC(CC)C(NC(C)=O)[C@@H]1[C@H](O)[C@@H](C(=O)O)C[C@H]1NC(=N)N]

To a 500.0-L reactor, 30.0 kg of (1S,2S,3R,4R)-3-[(S)-1-acetylamido-2-ethylbutyl]-4-guanidino-2-hydroxycyclopentane-1-carboxylic acid (peramivir) was added, and suspended in 240.0 L of purified water. The reaction product was heated at about 95° C. to completely dissolve the suspension, followed by natural cooling with stirring. When the temperature of the reaction product was 85° C., 45.0 L of 2-propanol was slowly added, followed by natural cooling to 25° C. with stirring. Then, peramivir trihydrate crystal grains were generated at about 40° C. When the temperature of the reactor reached 25° C. through natural cooling, stirring was carried out for about 12 hours for phase transition to crystal form A. In addition, the temperature of the reactor was cooled to 5° C., followed by further stirring for about 3 hours, and after the completion of the reaction, filtration was carried out and washing with 15.0 L of 10% 2-propanol solvent was carried out once. The filtered solid cake was placed in a vacuum dryer with an inner humidity of about 40% in which a purified water bowl was placed in the lower portion, and the solid cake was dried at 40° C. for about 12 hours. After the completion of the drying, peramivir trihydrate was obtained (yield: 95.5%, purity: 99.9%, and moisture: 14.6%).

¹H NMR (500 MHz, D₂O, δ): 4.30-4.27 (m, 2H), 3.79-3.74 (m, 1H, —CH), 2.65-2.63 (m, 1H, —CH), 2.48-1.45 (m, 1H, —CH), 2.16-2.12 (m, 1H, —CH), 1.88 (s, 3H, —CH₃), 1.75-1.71 (m, 1H, —CH), 1.41-1.36 (m, 3H), 0.95-0.91 (m, 2H), 0.87-0.84 (t, 3H, —CH₃), 0.82-0.79 (t, 3H, —CH₃); ¹³C NMR (500 MHz, D₂O, δ): 181.671, 173.686, 155.584, 75.368, 55.201, 54.091, 50.314, 49.933, 43.409, 34.015, 22.780, 21.887, 20.942, 12.128, 11.276; LC-MS (m/z, C₁₅H₂₈N₄O₄): calcd for 329.21, found; 329.5.

Table 1 below shows 2-theta (deg) values of crystal form A of peramivir trihydrate, which was a compound produced in Example 2.

TABLE 1

| | 2-theta | | Height | FWHM | Phase | Rel. int. I | Rel. height |
|---|---|---|---|---|---|---|---|
| No. | (deg) | d (ang.) | (cps) | (deg) | name | (a.u.) | (a.u.) |
| 1 | 4.655(6) | 18.97(3) | 8303(118) | 0.137(10) | Unknown | 100.00 | 100.00 |
| 2 | 6.001(4) | 14.715(10) | 5183(93) | 0.119(4) | Unknown | 37.75 | 62.43 |
| 3 | 6.499(2) | 13.588(4) | 2265(61) | 0.154(5) | Unknown | 22.21 | 27.27 |
| 4 | 7.577(7) | 11.658(11) | 1895(56) | 0.172(7) | Unknown | 22.44 | 22.83 |
| 5 | 8.899(6) | 9.928(6) | 1518(50) | 0.116(6) | Unknown | 11.95 | 18.28 |
| 6 | 9.3155(19) | 9.4858(19) | 4543(87) | 0.1282(17) | Unknown | 38.73 | 54.71 |
| 7 | 10.072(16) | 8.775(14) | 335(24) | 0.130(13) | Unknown | 2.47 | 4.03 |
| 8 | 11.11(5) | 7.96(3) | 87(12) | 0.20(4) | Unknown | 0.90 | 1.04 |
| 9 | 12.020(3) | 7.3568(17) | 2949(70) | 0.135(3) | Unknown | 25.72 | 35.51 |
| 10 | 12.303(3) | 7.1884(18) | 1132(43) | 0.133(6) | Unknown | 9.78 | 13.63 |
| 11 | 12.932(5) | 6.840(3) | 2225(61) | 0.221(10) | Unknown | 30.47 | 26.79 |
| 12 | 13.168(4) | 6.7181(19) | 1914(56) | 0.141(9) | Unknown | 16.73 | 23.05 |
| 13 | 13.611(7) | 6.500(3) | 769(36) | 0.175(16) | Unknown | 8.33 | 9.27 |
| 14 | 13.841(11) | 6.393(5) | 766(36) | 0.14(3) | Unknown | 6.45 | 9.23 |
| 15 | 13.992(9) | 6.324(4) | 1133(43) | 0.147(19) | Unknown | 10.32 | 13.65 |
| 16 | 14.498(14) | 6.105(6) | 403(26) | 0.21(3) | Unknown | 5.16 | 4.86 |
| 17 | 14.745(8) | 6.003(3) | 638(33) | 0.17(2) | Unknown | 6.68 | 7.68 |
| 18 | 15.299(8) | 5.787(3) | 3390(75) | 0.358(7) | Unknown | 75.12 | 40.82 |
| 19 | 15.923(14) | 5.561(5) | 185(18) | 0.16(4) | Unknown | 1.85 | 2.23 |
| 20 | 16.658(13) | 5.317(4) | 981(40) | 0.260(12) | Unknown | 15.29 | 11.81 |
| 21 | 17.131(11) | 5.172(3) | 534(30) | 0.174(19) | Unknown | 5.57 | 6.43 |
| 22 | 17.406(10) | 5.091(3) | 454(28) | 0.16(2) | Unknown | 4.46 | 5.47 |
| 23 | 17.854(11) | 4.964(3) | 307(23) | 0.15(2) | Unknown | 2.82 | 3.70 |
| 24 | 18.718(6) | 4.7367(16) | 2038(58) | 0.238(5) | Unknown | 30.19 | 24.55 |
| 25 | 19.575(8) | 4.5313(19) | 1340(47) | 0.131(15) | Unknown | 10.73 | 16.14 |
| 26 | 19.716(13) | 4.499(3) | 1308(47) | 0.16(3) | Unknown | 12.65 | 15.75 |
| 27 | 20.199(5) | 4.3926(11) | 3325(74) | 0.208(6) | Unknown | 42.11 | 40.05 |
| 28 | 20.737(6) | 4.2799(12) | 1995(58) | 0.219(8) | Unknown | 26.60 | 24.03 |
| 29 | 21.199(5) | 4.1876(10) | 1812(55) | 0.200(8) | Unknown | 22.09 | 21.82 |
| 30 | 21.766(16) | 4.080(3) | 963(40) | 0.140(12) | Unknown | 7.25 | 11.60 |
| 31 | 22.144(15) | 4.011(3) | 425(27) | 0.12(2) | Unknown | 2.71 | 5.11 |
| 32 | 22.831(7) | 3.8919(12) | 990(41) | 0.258(11) | Unknown | 13.97 | 11.93 |
| 33 | 23.256(7) | 3.8217(12) | 719(35) | 0.217(15) | Unknown | 8.52 | 8.66 |
| 34 | 23.770(7) | 3.7402(12) | 476(28) | 0.10(2) | Unknown | 2.71 | 5.73 |
| 35 | 24.158(7) | 3.6809(10) | 2767(68) | 0.276(6) | Unknown | 41.65 | 33.33 |
| 36 | 24.783(8) | 3.5895(12) | 759(36) | 0.237(17) | Unknown | 9.84 | 9.14 |
| 37 | 25.196(12) | 3.5316(16) | 415(26) | 0.30(3) | Unknown | 6.70 | 5.00 |
| 38 | 26.35(3) | 3.379(4) | 806(37) | 0.36(5) | Unknown | 15.65 | 9.70 |
| 39 | 26.584(8) | 3.3503(10) | 1116(43) | 0.17(2) | Unknown | 10.47 | 13.44 |
| 40 | 27.305(12) | 3.2635(14) | 403(26) | 0.32(3) | Unknown | 7.03 | 4.86 |
| 41 | 27.863(8) | 3.1993(9) | 1004(41) | 0.331(13) | Unknown | 17.96 | 12.09 |
| 42 | 28.457(13) | 3.1339(14) | 728(35) | 0.50(3) | Unknown | 19.55 | 8.77 |
| 43 | 29.659(9) | 3.0096(9) | 834(37) | 0.32(2) | Unknown | 14.57 | 10.04 |
| 44 | 30.025(7) | 2.9737(7) | 918(39) | 0.20(2) | Unknown | 10.09 | 11.06 |
| 45 | 30.308(7) | 2.9466(6) | 970(40) | 0.20(2) | Unknown | 10.41 | 11.68 |
| 46 | 30.658(7) | 2.9137(7) | 1527(50) | 0.276(12) | Unknown | 22.81 | 18.39 |
| 47 | 31.289(10) | 2.8564(9) | 699(34) | 0.287(14) | Unknown | 10.85 | 8.42 |
| 48 | 31.668(7) | 2.8230(6) | 530(30) | 0.120(19) | Unknown | 3.45 | 6.39 |
| 49 | 31.934(10) | 2.8002(9) | 463(28) | 0.23(2) | Unknown | 5.67 | 5.58 |
| 50 | 32.862(13) | 2.7232(11) | 173(17) | 0.13(3) | Unknown | 1.37 | 2.09 |
| 51 | 33.181(12) | 2.6977(9) | 386(25) | 0.26(3) | Unknown | 6.07 | 4.65 |
| 52 | 33.785(10) | 2.6509(8) | 604(32) | 0.244(14) | Unknown | 8.97 | 7.28 |
| 53 | 34.244(10) | 2.6164(8) | 332(24) | 0.20(2) | Unknown | 4.12 | 4.00 |
| 54 | 35.169(13) | 2.5496(9) | 467(28) | 0.20(2) | Unknown | 5.00 | 5.62 |
| 55 | 35.472(17) | 2.5286(12) | 307(23) | 0.30(8) | Unknown | 4.93 | 3.70 |
| 56 | 35.827(15) | 2.5043(10) | 252(20) | 0.20(5) | Unknown | 2.80 | 3.03 |
| 57 | 36.17(2) | 2.4813(13) | 398(26) | 0.26(3) | Unknown | 5.58 | 4.79 |
| 58 | 39.60(17) | 2.274(9) | 89(12) | 1.2(3) | Unknown | 6.63 | 1.07 |
| 59 | 40.13(4) | 2.245(2) | 87(12) | 0.29(15) | Unknown | 1.59 | 1.04 |
| 60 | 42.38(3) | 2.1312(12) | 139(15) | 0.11(6) | Unknown | 0.80 | 1.67 |
| 61 | 49.55(12) | 1.838(4) | 64(10) | 0.55(10) | Unknown | 1.84 | 0.77 |
| 62 | 59.62(3) | 1.5494(7) | 57(10) | 0.11(5) | Unknown | 0.60 | 0.69 |

The results of producing peramivir trihydrate by adjusting the amount of addition of 2-propanol from 40 L to 48 L for reactions are shown as follows (Table 2).

TABLE 2

| | | Volume | Hydrate | |
|---|---|---|---|---|
| Water | 2-Propanol | ratio | produced | Yield |
| 240.0 L | 40.0 L | 6:1 | Peramivir | ~94% |
| 240.0 L | 45.0 L | 5.3:1 | trihydrate | 95.5% |
| 240.0 L | 48.0 L | 5:1 | | ~92% |

### Example 3: Preparation of Peramivir Trihydrate ((1S,2S,3R,4R)-3-[(S)-1-acetylamido-2-ethyl butyl]-4-guanidino-2-hydroxycyclopentane-1-carboxylic Acid Trihydrate) (3)

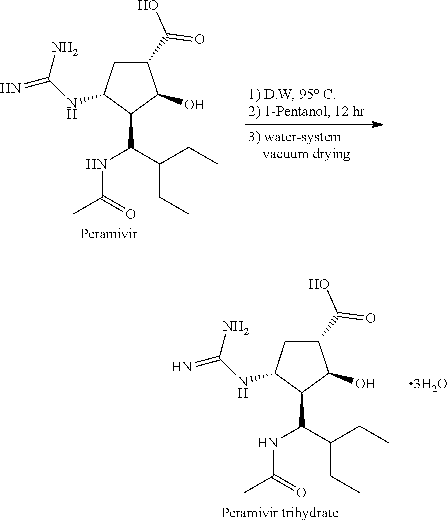 [SMILES: CCC(CC)C(NC(C)=O)[C@@H]1[C@H](O)[C@@H](C(=O)O)C[C@H]1NC(=N)N ; CCC(CC)C(NC(C)=O)[C@@H]1[C@H](O)[C@@H](C(=O)O)C[C@H]1NC(=N)N]

To a 500.0-L reactor, 30.0 kg of (1S,2S,3R,4R)-3-[(S)-1-acetylamido-2-ethylbutyl]-4-guanidino-2-hydroxycyclopentane-1-carboxylic acid (peramivir) was added, and suspended in 240.0 L of purified water. The reaction product was heated at about 95° C. to completely dissolve the suspension, followed by natural cooling with stirring. When the temperature of the reaction product was 85° C., 45.0 L of 1-pentanol was slowly added, followed by natural cooling to 25° C. with stirring. Then, peramivir trihydrate crystal grains were generated at about 40° C. When the temperature of the reactor reached 25° C. through natural cooling, stirring was carried out for about 12 hours for phase transition to crystal form A. In addition, the temperature of the reactor was cooled to 5° C., followed by further stirring for about 3 hours, and after the completion of the reaction, filtration was carried out and washing with 15.0 L of 10% 1-pentanol solvent was carried out once. The filtered solid cake was placed in a vacuum dryer with an inner humidity of about 40% in which a purified water bowl was placed in the lower portion, and the solid cake was dried at 40° C. for about 12 hours. After the completion of the drying, peramivir trihydrate was obtained (yield: 95.1%, purity: 99.9%, and moisture: 14.1%).

¹H NMR (500 MHz, D₂O, δ): 4.30-4.27 (m, 2H), 3.79-3.74 (m, 1H, —CH), 2.65-2.63 (m, 1H, —CH), 2.48-1.45 (m, 1H, —CH), 2.16-2.12 (m, 1H, —CH), 1.88 (s, 3H, —CH₃), 1.75-1.71 (m, 1H, —CH), 1.41-1.36 (m, 3H), 0.95-0.91 (m, 2H), 0.87-0.84 (t, 3H, —CH₃), 0.82-0.79 (t, 3H, —CH₃); ¹³C NMR (500 MHz, D₂O, δ): 181.671, 173.686, 155.584, 75.368, 55.201, 54.091, 50.314, 49.933, 43.409, 34.015, 22.780, 21.887, 20.942, 12.128, 11.276; LC-MS (m/z, C₁₅H₂₈N₄O₄): calcd for 329.21, found; 329.5.

1. A method for producing (1S,2S,3R,4R)-3-[(S)-1-acetylamido-2-ethylbutyl]-4-guanidino-2-hydroxycyclopentane-1-carboxylic acid trihydrate (peramivir trihydrate) represented by chemical formula 2: 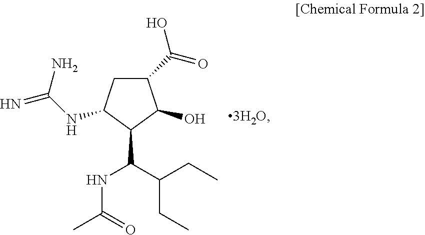 [SMILES: CCC(CC)C(NC(C)=O)[C@@H]1[C@H](O)[C@@H](C(=O)O)C[C@H]1NC(=N)N]
the method comprising:
dissolving (1S,2S,3R,4R)-3-[(S)-1-acetylamido-2-ethylbutyl]-4-guanidino-2-hydroxycyclopentane-1-carboxylic acid (peramivir) in water to prepare an aqueous solution; and
adding an alcoholic solvent selected from 1-propanol, 2-propanol, 1-pentanol, 2-butanol, 3-methyl-1-butanol, 2-methyl-1-propanol, or a mixture thereof.

2. The method of claim 1, wherein the peramivir and water are added such that the ratio of the weight of water to the weight of peramivir is 1:1 to 1:30.

3. The method of claim 1, wherein the dissolving is conducted with heating and the heating is conducted at a temperature of 90 to 95° C.

4. The method of claim 1, wherein the alcoholic solvent is added at 75 to 90° C.

5. The method of claim 1, wherein the alcoholic solvent is added at a volume ratio, relative to the volume of water, of 1:1-10 (alcoholic solvent:water).

6. The method of claim 1, further comprising cooling the aqueous solution with the alcoholic solvent added thereto, to a temperature of 15 to 30° C.

7. The method of claim 6, further comprising performing stirring for 6 to 24 hours after the cooling.

8. The method of claim 7, further comprising performing stirring with cooling to a temperature of 1 to 10° C. after the stirring.

9. The method of claim 6, further comprising filtering peramivir trihydrate crystals generated by the cooling to prepare a peramivir trihydrate crystal cake.

10. The method of claim 9, further comprising washing the peramivir trihydrate crystal cake with an alcoholic aqueous solution.

11. The method of claim 10, wherein the alcoholic aqueous solution is an aqueous solution of 1-propanol, an aqueous solution of 2-propanol, an aqueous solution of 1-pentanol, an aqueous solution of 2-butanol, an aqueous solution of 3-methyl-1-butanol, an aqueous solution of 2-methyl-1-propanol, or a mixture thereof.

12. The method of claim 10, wherein the concentration of the alcoholic aqueous solution is 1 to 30%.

13. The method of claim 10, wherein the volume of the alcoholic aqueous solution is a volume corresponding to 0.1 to 20 times the initial weight of peramivir.

14. The method of claim 1, further comprising subjecting a product, generated through the adding of the alcoholic solvent, to vacuum drying or decompression drying.

15. The method of claim 14, wherein the drying is conducted in a temperature condition of 25 to 80° C.

16. The method of claim 14, wherein the drying is humidification drying in a humidity condition of 20 to 80%.
